(19) United States
(12) Patent Application Publication
(10) Pub. No.: US 2025/0087302 A1
(43) Pub. Date: Mar. 13, 2025

(54) MACHINE LEARNING MODELS FOR CELL LINE SELECTION

(71) Applicant: Hoffmann-La Roche Inc., Little Falls, NJ (US)

(72) Inventors: Shan-Hua CHUNG, Muenchen (DE); Daniel Tobias GROSSKOPF, Muenchen (DE); Styliani PAPADAKI, Muenchen (DE); Oliver Helmut POPP, Penzberg (DE); Tom Kamran QUAISER, Penzberg (DE); Laura Susanne STOECKL, Penzberg (DE); Styliani TOURNAVITI, Muenchen (DE)

(73) Assignee: Hoffmann-La Roche Inc., Little Falls, NJ (US)

(21) Appl. No.: 18/830,789

(22) Filed: Sep. 11, 2024

(30) Foreign Application Priority Data
Sep. 12, 2023 (EP) 23196981.7

(51) Int. Cl. G16B 20/20; G16B 40/20

(52) U.S. Cl. CPC G16B 20/20; G16B 40/20

(57) ABSTRACT

Methods for facilitating selection of cell lines for production of recombinant proteins are disclosed. In particular, disclosed is the use of machine learning models trained on multiomics data to predict one or more values indicative of the titre and/or quality of a recombinant protein expressed by different cell lines, enabling ranking the cell lines based on the predicted values and selecting those predicted to produce the recombinant protein with higher titre and/or higher quality.

### FIELD OF THE DISCLOSURE

The present invention relates to methods for facilitating selection of cell lines for production of recombinant proteins. In particular, the present invention relates to the use of machine learning models trained on multiomics data to predict one or more values indicative of the titre and/or quality of a recombinant protein expressed by different cell lines, enabling ranking the cell lines based on the predicted values and selecting those predicted to produce the recombinant protein with higher titre and/or higher quality.

### BACKGROUND

Recombinant proteins, e.g. monoclonal antibodies (mAbs) are considered one of the most game-changing products of the biopharmaceutical industry [1]. Being on the market for the past 36 years, they have found applications in several therapeutic areas and demonstrate continuous commercial power growth [2]. Even though production of standard monoclonal antibodies is no longer considered to be problematic for the biopharmaceutical industry, the current entry of the “difficult to express” (DTE) mAbs formats demand a continuous improvement of the industrial production pipelines [3]. Chinese hamster ovary (CHO) cells are currently representing the most predominant host cell lines for the production of therapeutic proteins [1]. A typical cell line development (CLD) process of biomanufacturing, starts with transfection of host cells, e.g. CHO host cells, with the plasmid vectors containing the multiple candidate sequences [4,5]. After transfection, the recovering cell culture is split into several master pools, from which the most stable are chosen for single cell cloning [6]. Cell lines originating from a single cell (hereafter referred to as clones, or cell clones) are screened for growth, productivity and product quality and only high producer clones are being progressively scaled-up from batch to fed-batch conditions [7,8] (FIG. 1). While in controlled mini-bioreactors systems, they are evaluated for antibody titre and product quality, as well as for cell clone growth and stability, until a lead clone is selected as the “best” producer clone [9,10]. Once conditions and the lead clone are defined, the process is then transferred to a large-scale production to generate material for preclinical and clinical supply, under current good manufacturing practices (cGMP) regulations [11].

As such, the selection of the lead clone typically requires 6 to 12 months and is a time, resources and labour intensive process [4,11,12]. Thus, the biopharmaceutical industry is continuously looking for innovative solutions that will speed-up drug development while maintaining the desired cell line productivity attributes [13]. Identification of highly expressing cell clones is a critical process and requires effective screening methods during the whole CLD and process development pipeline. [14]. Particularly, screening clonal profiles shortly after single cell cloning (primary clonal screening), where the cell clones are characterized by low density, requires methods offering high specificity, simplicity, stability, and rapid analysis [15]. To achieve the throughput required for screening thousands of clones for high titres and the desired product quality, traditional immunoassays such as enzyme-linked immunosorbent assays (ELISA) have become a commonly used tool as a primary clonal screen [16]. However, using these assays, each mAb campaign requires significant assay development and validation effort up front. Additionally, this primary clonal screening is based on assays that take into consideration only the productivity phenotype of each done and no other biological information [12,14,17]. However, there are many published examples demonstrating the value of omics data by providing targets (e.g. genes, metabolites etc.) directly associated with the clonal phenotype of interest [18-21]. Still, identification of a minimal set of targets driving the phenotype and deployment strategies of those into a mAb campaign are actionables to be fulfilled [22].

Meanwhile, recent advancements in artificial intelligence (AI) and machine learning (ML) technologies together with improved large-scale data capturing have set the stage for a synergistic framework, in which the predictive capabilities of AI/ML can lead to transformative improvements in the process development field [23-25]. The automated nature of model-based methods makes them an attractive choice to use as approximations for experimental models that are expensive or difficult to evaluate. Despite that, only a few studies describing ML-based prediction of attributes such as titre, product quality, and growth have been published [26]. One of those was published by Povey et al., demonstrating the benefit of combining mass spectrometry and PLS-DA modelling for the selection of recombinant mammalian cell lines [27]. In particular, they developed a predictive model to forecast the productivity of unknown cell clones at the 10 L scale based on MALDI-ToF fingerprint at the 96 deep well plate scale. Clarke et al. presented a predictive model of productivity in CHO bioprocess culture based on gene expression profiles. They constructed a model consisting of 287 genes, capable of predicting cell specific productivity with high accuracy [28]. Furthermore, recently Barberi et al. [29,30] predicted the monoclonal antibody end-point titre using metabolomics information measured during the Ambr15 mini bioreactor fermentation process.

Yet, the potential of model-based methods in the field is still not fully exhausted [31]. One of the main caveats of ML applications in biomanufacturing is data availability [26,32,33]. Commonly, the data provided for building statistical modelling methodologies to predict the process development outcome are derived only from a small sample size. Due to time and cost constraints, relevant measurements and analytics are only taken from a single molecule development batch. Therefore, this has a limited chance to extrapolate to more complex formats and even completely different bioprocesses [34]. In statistical modelling input features are the ones the model is trained on to explain or predict changes in the target variables [35]. In the current modelling approaches not only the sample sizes, but also the input features are limited. Measurements comprise standard technologies that are already part of the process development pipeline [36,37], but do not go beyond. Ultimately, such data-driven methods have rarely been applied to study the relationship between productivity attributes and molecular components [38].

Therefore, it is desirable to provide robust models to facilitate selection of a cell line among a multiplicity of cell lines, predicted to produce the recombinant protein of interest with high titre and/or quality, in early stages of CLD.

### SUMMARY OF THE DISCLOSURE

Broadly, the present invention provides methods for facilitating selection of a subset of mammalian cell lines, among several candidate cell lines, which are predicted to express a recombinant protein of interest in high titres and/or high quality compared to the other candidate cell lines.

Accordingly, a first aspect provides a computer-implemented method for facilitating selection of a cell line, from among a plurality of candidate cell lines that produce a recombinant protein, the method comprising:
(a) receiving omics data for each of the plurality of candidate cell lines; and
(b) using a machine learning model to predict one or more values indicative of recombinant protein titre and/or quality,
wherein the machine learning model has been trained using a training dataset comprising omics data for a multiplicity of cell lines, and for each cell line, one or values indicative of recombinant protein titre and/or quality, and
wherein the omics data comprises: i) transcriptomics data; ii) metabolomics data; iii) proteome data; iv) transcriptomics and metabolomics data; v) transcriptomics and proteome data; vi) metabolomics and proteome data; or vii) transcriptomics, metabolomics, and proteome data.

Also described herein according to a second aspect is a computer program product, comprising computer readable instructions which, when executed by one or more processors, cause the one or more processors to carry out the method of the first aspect or any embodiments thereof.

Also described herein according to a third aspect is a non-transitory computer-readable medium having stored thereon computer readable instructions which, when executed by one or more processors, cause the one or more processors to carry out the method of the first aspect or any embodiments thereof.

Also described herein according to a fourth aspect is a system, comprising: at least one processor; and at least one non-transitory computer readable medium containing instructions that, when executed by the at least one processor, cause the at least one processor to perform the method of the first aspect or any embodiments thereof.

### BRIEF DESCRIPTION OF THE FIGURES

FIG. 1 depicts cell line development, from pool generation until monoclonal cell line evaluation in the Ambr15 fermentation system. FIG. 1a schematic and FIG. 1b more detailed.

FIG. 2 shows the methodology of Example 1.

FIG. 3 shows an example of original versus interpolated measurements of one sample during fermentation process.

FIG. 4 shows per-project distribution of titre variables.

FIG. 5 shows per-project distribution of standardized titre variables.

FIG. 6 shows stratified K-fold cross validation and testing set up. K=Project 01-09.

FIG. 7 shows pairwise comparison of the prediction performance between baseline and metabolomics model in the per project performance prediction set up, using paired Wilcoxon tests (ns=P>0.05, *=P≤0.05, **=P≤0.01, ***=P≤0.001). Due to the absence of the baseline values for Project 5, the predictions of it were not added in the plot for the product titre variables MP CESDS, Eff. Titer SECMS, Eff. Titer Cedex Day 14, Eff. Titer Protein A. However, the project data were used in the total dataset for the prediction of the rest of the projects. (R² of Project 5 prediction: MP CESDS=0.12, Eff. Titer SECMS=0.43, Eff. Titer Cedex Day 14=0.41, Eff. Titer Protein A=0.44).

FIG. 8 shows pairwise comparison of the prediction performance between baseline and RapidFire-MS model in the per project performance prediction set up, using paired Wilcoxon tests (ns=P>0.05, *=P≤0.05, **=P≤0.01, ***=P≤0.001).

FIG. 9 shows pairwise comparison of the prediction performance between baseline and transcriptomics model in the per project performance prediction set up, using paired Wilcoxon tests (ns=P>0.05, *=P≤0.05, **=P≤0.01, ***=P≤0.001). Due to the absence of the baseline values for Project 5, the predictions of it were not added in the plot for the product titer variables MP CESDS, Eff. Titer SECMS, Eff. Titer Cedex Day 14, Eff. Titer Protein A. However, the project data were used in the total dataset for the prediction of the rest of the projects. (R² of Project 5 prediction: MP CESDS=0.55, Eff. Titer SECMS=0.39, Eff. Titer Cedex Day 14=0.38, Eff. Titer Protein A=0.33).

FIG. 10 shows heat map of all the mean R² values generated from each single and multi assay model. On the x-axis are all the product titer variables that are predicted and on the y-axis all the different input features of the single and multi assay models. Due to the inconsistency of samples (absence of features for whole projects) between the single assay and the multi assay models, the baseline R² is calculated as an average of all the individual models per product titer variable. Additionally, due to the absence of the baseline values for Project 5, the predictions of it were not added in the plot for the product titer variables MP CESDS, Eff. Titer SECMS, Eff. Titer Cedex Day 14, Eff. Titer Protein A.

FIG. 11 shows pairwise comparison of the prediction performance between Baseline and Metabolomics-RapidFire model in the per project performance prediction set up, using paired Wilcoxon tests (ns=P>0.05, *=P≤0.05, **=P≤0.01, ***=P≤0.001). Projects 5 and 8 were excluded from the training set, due to absence of rapidFire-MS and Metabolomics measurements respectively.

FIG. 12 shows pairwise comparison of the prediction performance between Baseline and Metabolomics-Transcriptomics model in the per project performance prediction set up, using paired Wilcoxon tests (ns=P>0.05, *=P≤0.05, **=P≤0.01, ***=P≤0.001). Project 8 was excluded from the training set, due to absence of metabolomics measurements. Due to the absence of the baseline values for Project 5, the predictions of it were not added in the plot for the product titer variables MP CESDS, Eff. Titer SECMS, Eff. Titer Cedex Day 14, Eff. Titer Protein A. However, the project data were used in the total dataset for the prediction of the rest of the projects. (R² of Project 5 prediction: MP CESDS=0.56, Eff. Titer SECMS=0.57, Eff. Titer Cedex Day 14=0.56, Eff. Titer Protein A=0.49)

FIG. 13 shows pairwise comparison of the prediction performance between Baseline and RapidFire-Transcriptomics model in the per project performance prediction set up, using paired Wilcoxon tests (ns=P>0.05, *=P≤0.05, **=P≤0.01, ***=P≤0.001). Project 5 was excluded from the training set, due to absence of rapidFire-MS measurements.

FIG. 14 shows pairwise comparison of the prediction performance between Baseline and Metabolomics-RapidFire-Transcriptomics model in the per project performance prediction set up, using paired Wilcoxon tests (ns=P>0.05, *=P≤0.05, **=P≤0.01, ***=P≤0.001). Projects 5 and 08 were excluded from the training set, due to absence of rapidFire-MS and Metabolomics measurements respectively.

FIG. 15 shows T-test comparison of the mean actual effective titre measurements of the top 12 clones as predicted by the ML model (metabolomics, rapidFire-MS, transcriptomics) versus the rest of the clones. Projects 01, 02, 03, 04, 06, 07, 09.

FIG. 16 shows T-test comparison of the mean actual effective titre measurements of the top 12 clones as predicted by the ML model (metabolomics, transcriptomics) versus the rest of the clones. Projects 5.

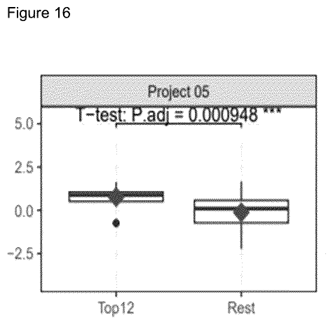

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Further, as used in the following, the terms “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention can, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

A compound as described herein may be a small molecule (e.g. a small molecule inhibitor, activator, cofactor, etc.) or a large molecule (e.g. a biologic, therapeutic protein or peptide such as an antibody or compound derived therefrom, a nucleic acid, etc.). A compound may be an organic compound. A compound may be a pharmaceutically active agent (also referred to as a drug or therapeutic agent), or a degradation product thereof.

The systems and methods described herein may be implemented in a computer system, including or in addition to the structural components and user interactions described. As used herein, the term “computer system” includes the hardware, software and data storage devices for embodying a system or carrying out a method according to the above-described embodiments. For example, a computer system may comprise a processing unit such as a central processing unit (CPU) and/or graphics processing unit (GPU), input means, output means and data storage, which may be embodied as one or more connected computing devices. Preferably, the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer.

The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

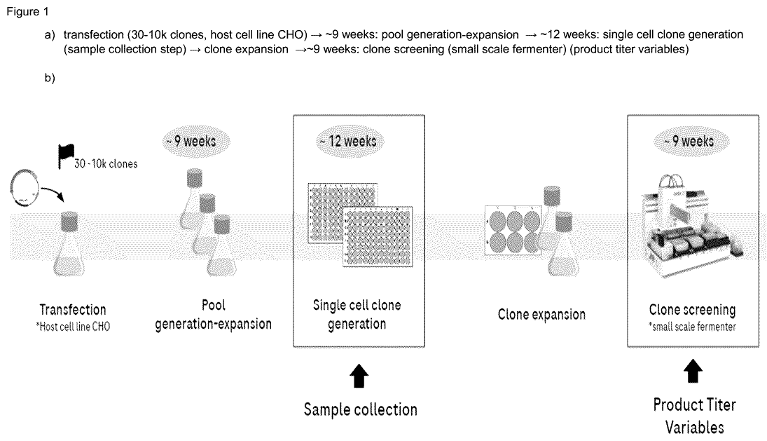

FIG. 1 shows the schematic representation of a typical cell line development (CLD) in biomanufacturing. It starts with transfection of host cell lines, e.g. CHO cells, with the DNA constructs (e.g. plasmid vectors) encoding multiple candidate sequences translating into the recombinant protein (e.g. chains of the antibody) of interest. At this stage, the transfected cell lines are called pools. For the development of one recombinant protein, e.g. an antibody, several plasmid vectors are constructed. The vectors are varying on the final molecule format translated out of the sequences. For example, in case of an antibody, within the vectors that are translating into the same format, there is additional variability in the chain ratios and the chain configuration. This means that for a particular antibody to be developed, several formats and several chain ratios and configurations within each format are being examined at the pool level. To identify highly productive and stable pools to be used for the rest of the cell line development process, all the different cell culture pools need to be expanded and further evaluated on stability and production rate. Thus, the pool assessment process is a time-consuming (˜8 weeks) and resource expensive activity. Out of this process, the final pools of a particular format and chain ratio and configuration are selected and they are used as starting material for single cell cloning generation (single cell cloning requires another ˜4 weeks). Monoclonal cell lines originating from a single cell (one cell separated from the pool in each well of a 96 well plate) are screened for growth, productivity and product quality and only high producer clones are being progressively scaled-up from batch to fed-batch conditions. As such, highly productive pools will result in highly productive monoclonal cell lines. While in controlled mini-bioreactors systems (e.g. Ambr15 bioreactor on the far right of the figure), they are evaluated for antibody titre and product quality, until a lead done is selected as the “best” producer done. The process for finding the best clone(s) is therefore very time- and resource-consuming.

As the first aspect, the present invention provides a computer-implemented method for facilitating selection of a cell line, from among a plurality of candidate cell lines that produce a recombinant protein, the method comprising:
(a) receiving omics data for each of the plurality of candidate cell lines; and
(b) using a machine learning model to predict one or more values indicative of recombinant protein titre and/or quality,
wherein the machine learning model has been trained using a training dataset comprising omics data for a multiplicity of cell lines, and for each cell line, one or values indicative of recombinant protein titre and/or quality, and
wherein the omics data comprises: i) transcriptomics data; ii) metabolomics data; iii) proteome data; iv) transcriptomics and metabolomics data; v) transcriptomics and proteome data; vi) metabolomics and proteome data; or vii) transcriptomics, metabolomics, and proteome data.

The methods of the present aspect may have any of the features described in relation to any other aspect.

The terms “host cell”, “host cell line”, “host cell culture”, and “cell lines” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

From an industrial perspective, the disclosed computer-implemented method can be used to assess the expressibility of the recombinant protein of interest by different cell clones during the cell line screening process. By applying the machine learning model disclosed herein, the expressibility and productivity of the cell lines could be evaluated easier and earlier. The process can be optimized by examining only the cell lines that are predicted to produce the recombinant protein with highest titres and/or quality. In that way, one can significantly reduce the occupation time of the bioreactor system, freeing up capacity for screening more molecules or just saving time and resources. This brings about a drastic increase in the throughput and possibilities of representative bioreactor performance prediction compared to techniques and workflows in the state of the art.

In an embodiment, the proteome data comprises data indicative of the titre of the main product and side products in the cell culture supernatant of the respective cell line.

In recombinant protein production, in particular when the protein comprises more than one polypeptide chains (e.g. antibodies), the main product is considered the protein with correct assembly of the polypeptides. Side products, on the other hand, are considered to be proteins composed of an undesired assembly of the polypeptides, though they comprise or are derived from the transgene.

In an embodiment, the omics data corresponding to each cell line in the training dataset is obtained in cell line development process at least one week, e.g. at least two, three, four, or five weeks, of cell culturing, before obtaining values indicative of recombinant protein titre and/or quality. Preferably, said data is measured from the samples (e.g. cell pellets and/or culture media) when the cells are in early stage of culture after cell sorting (e.g. by FACS or dilution) from the initial transfected cell pools, and before clone expansion and small-scale fermentation process (e.g. Ambr15 bioreactor in FIG. 1). The cells are cultured, usually in small volumes, such as in 24-well or 96-well plates, for at least 10-14 days. In an embodiment, the omics data is obtained during day 10-14 of said culturing.

In an embodiment, the values indicative of recombinant protein titre and/or quality are obtained during small-scale fermentation.

In an embodiment, the omics data of each cell line is obtained at the same time, e.g. from one common cell pellet or cell culture supernatant sample. For instance, on day 14 of single-cell culture, the culture media and cells are harvested together, and each one is used for their respective measurements (e.g. cell pellet is used for generating transcriptomics data, and the cell culture media, a.k.a. supernatant) is used for measurements related to proteome and metabolomics data.

In an embodiment, the metabolomics data comprise values corresponding to the concentration of C-nutrient source, N-nutrient source, anions, cations, recombinant protein (e.g. IgG) product, organic acids, total protein, amino acids, amino acid derivatives, vitamins, vitaminoids, metabolic breakdown products, organic acids, amines, formate, pyridoxamine, asymmetric dimethylarginine, methionine sulfoxide, alanin, lactic acid, ethanolamine, pyruvic acid, acetate, glycine, isoleucine, Tin and Vanadium, and/or other chemical elements, in cell culture supernatant. In an embodiment, the recombinant protein is an IgG antibody and the metabolomics data comprise values corresponding to the concentration of IgG, Formate, Pyridoxamine, Asymmetric dimethylarginine, Methionine Sulfoxide, Alanin, Lactic acid, Ethanolamine, Pyruvic acid, Acetate, Glycine, Isoleucine, Tin and Vanadium, in cell culture supernatant.

In an embodiment, the metabolomics data is obtained by one or methods selected from a group consisting of: i) ultra-high performance liquid chromatography tandem mass spectrometry method (LC-MS), preferably after protein precipitation; ii) single quadrupole inductively coupled plasma mass spectrometry (ICP-MS); and iii) Cedex Bio HT Analyzer. Details for exemplary methods, assays and measurements of the data are provided in Example 1.

In an embodiment, LC-MS is used for measuring the concentration of cell culture media components and metabolites, and/or ICP-MS is used for measuring the concentration of trace elements in cell culture supernatants.

Details for exemplary methods, assays and measurements of the data are provided in Example 1.

In an embodiment, the metabolomics data is preprocessed by dividing the values, e.g. the concentration of each metabolite, by the viable cell density and the average cell volume of the corresponding cell culture at the time of harvesting.

In an embodiment, the proteome data is obtained via mass spectrometry, e.g. high throughput RapidFire-mass spectrometry.

In an embodiment, before obtaining the proteome data the supernatants are pre-treated by removal of cell media and recombinant protein enrichment.

In an embodiment, the transcriptomics data is obtained from a cell pellet, preferably by a high-throughput method, e.g. RNA-seq.

In an embodiment, the one or more values indicative of recombinant protein titre and/or quality comprises the recombinant protein titre measured on day 10 (±half day), day 12 (±half day), and/or day 14 (±half day) of the fed batch culture, optionally by a Cedex Bio HT Analyzer.

In an embodiment, the one or more values indicative of recombinant protein titre and/or quality comprises the recombinant protein titre measured by analytical Protein A chromatography, preferably on day 14 (±half day) of the fed batch culture.

In an embodiment, the one or more values indicative of recombinant protein titre and/or quality comprises the percentage of correctly assembled recombinant protein, measured preferably on day 14 (±half day) of the fed batch culture, e.g. by capillary electrophoresis sodium dodecyl sulphate (CE-SDS).

In an embodiment, the one or more values indicative of recombinant protein titre and/or quality comprises the titre of the main product, measured preferably on day 14 (±half day) of the fed batch culture, e.g. by quantitative size exclusion liquid chromatography coupled with mass spectrometry (qSEC-MS).

In an embodiment, the one or more values indicative of recombinant protein titre and/or quality is calculated by multiplying the value of recombinant protein titre as measured according to claim 14 by the percentage of correctly assembled recombinant protein as measured according to claim 15, wherein both measurements have been performed on the same day, preferably day 14 of the fed batch culture.

In an embodiment, the one or more values indicative of recombinant protein titre and/or quality is calculated by multiplying the value of recombinant protein titre as measured according to claim 13 by the percentage of correctly assembled recombinant protein as measured according to claim 15, wherein both measurements have been performed on the same day, preferably day 14 of the fed batch culture.

In an embodiment, the cells are mammalian cells, e.g. CHO cells.

In an embodiment, the recombinant protein is an antibody (e.g. an IgG antibody) or a fragment thereof.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv, and scFab); single domain antibodies (dAbs); and multispecific antibodies formed from antibody fragments. For a review of certain antibody fragments, see Holliger and Hudson, Nature Biotechnology 23:1126-1136 (2005).

In an embodiment, the amino acid sequence of the recombinant protein expressed by the plurality of candidate cell lines is the same.

In an embodiment, the machine-learning model comprises regression analysis, preferably a random forest regression model.

In an embodiment, the computer-implemented method of aspect 1 or any embodiments thereof further comprise ranking the cell lines according to the predicted one or more values indicative of recombinant protein titre and/or quality, wherein the cell lines with higher predicted values are advanced to a next step of cell line screening or fermentation, e.g. a fed batch cell culture stage.

In an embodiment, the method comprises presenting to a user an indication of the ranking of the cell lines.

In another aspect, a computer program product is disclosed, comprising computer readable instructions which, when executed by one or more processors, cause the one or more processors to carry out the method of any of embodiments of any method described herein.

In another aspect, a non-transitory computer-readable medium is disclosed, having stored thereon computer readable instructions which, when executed by one or more processors, cause the one or more processors to carry out the method of any of embodiments of any method.

In another aspect, a system is disclosed, comprising: at least one processor; and at least one non-transitory computer readable medium containing instructions that, when executed by the at least one processor, cause the at least one processor to perform the method of any of embodiments of any method.

The various concepts introduced above and discussed in greater detail below may be implemented in any of numerous ways, and the described concepts are not limited to any particular manner of implementation. Examples of implementations are provided for illustrative purposes. The following is presented by way of example and is not to be construed as a limitation to the scope of the claims.

### EXAMPLES

### Example 1

Here we present a rich and comprehensive CHO omics dataset of 892 randomly selected cell clones producing 11 different therapeutic antibody formats. The data consists of metabolomics data, transcriptomics data, and antibody proteome measured by rapidFire-MS, collected during the primary clonal screening after single cell cloning in cell line development (CLD). We utilized a random forest regression algorithm, for each individual omics assay as well as combinations of them, in a format-fold cross validation set-up to predict antibody production attributes of the later performed process in fed-batch bioreactors. The final multi-omics model demonstrated a statistically significant higher predictive capability compared to a baseline model. Additionally the models combining more than one omics assay showed higher prediction rate to the individual, indicating that combination of multiple omics groups can better replicate the cell clones production performance. This approach sets the basis for a very early prediction of cell clones' performance and the foundation for a faster and more robust in silico based CLD.

### Materials and Methods

### Methodology

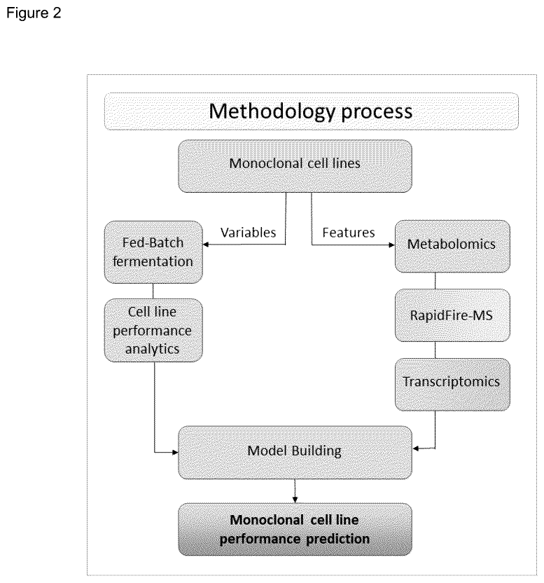

As shown in FIG. 2, hundreds of CHO cell clones were randomly selected and during the primary clonal screening, after the single cell cloning procedure. Different sample aliquots were distributed to the different assays. Particularly, per cell clone, three cell-free supernatant samples were used for the three different metabolomics assays, one cell-free supernatant was used for the RapidFire-MS analysis and another with cell pellets was used for the RNA sequencing analysis. All the aliquots were harvested on the same day. Each omics assay (metabolomics, proteome (e.g. RapidFire-MS), transcriptomics) is followed by corresponding data preprocessing and feature engineering. Additionally, a fourth sample of cell culture was used for further batch and fed-batch fermentation followed by cell clone performance analytics. This study focuses on the main body of FIG. 2, the “Monoclonal cell line performance prediction”. In short, the preprocessed data per assay are used as input features for the model to be trained on and the cell line analytics as target variables to be predicted. The trained model should utilize the biological information provided by the omics features to predict future productivity behaviour of the cells. We built several ML models, consisting of a single omics assay and several combinations of them. In such a manner, we assessed the predictive capability of each one of the assays individually, as well as the additive value offered by the combinations of different omics assays.

### Monoclonal CHO Cell Lines Culture

All cell clones were generated using the CHO Host Cell Line (International patent publication number WO 2019/126 634 A2) [39]. Pools of cells that stably express the therapeutic proteins were generated as described in [40]. The cell clones producing the 11 different therapeutic protein formats have been cultivated each cultivated in batches of 87-96 samples, one in each well in 4×24 deep well plates. Each batch contains cell clones derived from 2-3 different pools expressing different gene configurations of the same protein format. For simplicity, all the clones producing the same format are referred hereafter as Projects. Table 1, shows the whole sample collection including, the format produced per project and the number of clones. The clones from each Project were randomly selected after single cell cloning by limited dilution and cultivated in proprietary medium in 24 deep well plates at 350 rpm, 37° C., 85% rH and 5% CO₂. Random selection of the cell clones provided an unbiased variety of clone performances for the model to be trained on, resulting in a model capable of predicting a wider range of productivity performances. Cells were passaged three times at a seeding density of 3×10\hat{ }5 cells/ml every 3-4 days. During the third passage, cell banking was performed, saving 3 frozen vials per cell clone. Three days after the last passage, pellets and supernatant from each cell clone were harvested for the transcriptomics and the metabolomics and rapidFire-MS measurements, respectively. In total 1009 samples were collected. We strictly isolated a hold-out dataset for final testing of the model, after training and validation, where our model should predict unseen data from new therapeutic proteins. For training and validation of the models Project 1-9 were used and for testing Projects 10 and 11.

TABLE 1
Types of therapeutic proteins collected
and number of samples per batch.

| | | Number of | |
|---|---|---|---|
| Batch | | transfection | Number of |
| Name | Therapeutic protein type | pools | cell clones |
| Project 01 | 2 + 1 T cell bispecific antibody | 2 | 93 |
| Project 02 | 2 + 1 T cell bispecific antibody | 2 | 82 |
| Project 03 | 1 + 1 bispecific antibody | 2 | 89 |
| Project 04 | 1 + 1 ectodomain fusion antibody | 2 | 96 |
| Project 05 | 2 + 1 brain shuttle antibody | 2 | 96 |
| Project 06 | 3 + 1 bispecific antibody | 3 | 91 |
| Project 07 | bispecific dutaFab | 2 | 96 |
| Project 08 | pentamer protein | 2 | 96 |
| Project 09 | generic antibody | 2 | 95 |
| Project 10 | 2 + 1 T cell bispecific antibody | 2 | 94 |
| Project 11 | 2 + 1 bispecific antibody | 2 | 81 |

### Seed-Train and Fed-Batch Production Assay

One batch of cell banking vials was used for a 4 weeks manual seed-train cultivation followed by fed-batch fermentation. Cells were cultured in 125 ml shake flasks at 150 rpm, 37° C., 70% rH and 5% CO₂ and were passaged at a seeding density of 3-14×10\hat{ }5 cells/ml every 3-4 days. Fed-batch cultures were performed in the Ambr15 miniature bioreactor system (Sartorius Stedim Biotech, Sartorius AG) with chemically-defined production media. Cells were seeded with 8×10\hat{ }6 cells/ml on day 0 of the production stage after adaptation to production media during two passages. Cultures received daily feed medium after day 3 and additional feed bolus on days 3, 7 and 10. Cells were cultivated for 14 days. Cultivation in the ambr15 system was operated at set points of 36° C., DO 30%, pH 7.0 and an agitation rate of 1200 rpm. After the above several cultivation steps, out of the initial 1009 cell clones, 892 survived and were used for this study.

### Cell Clones Analytics

For the final cell line characterization, 4 different productivity evaluation assays were performed. Product titre was measured from supernatant harvested from the fed-batch fermentation on days 10, 12 & 14 using Cedex Bio HT Analyzer (Roche CustomBiotech, F. Hoffmann-La Roche Ltd). Additionally, titre was measured by analytical protein A chromatography and ultra high performance liquid chromatography with ultraviolet (UV) light detection (Dionex Ultimate 3000 UHPLC fitted with POROS A 20 μm Column, Thermo Fisher Scientific Inc.). Product quality was assessed as percentage of correctly assembled protein (main peak on the chromatogram) and was measured by capillary electrophoresis sodium dodecyl sulphate (CE-SDS; HT Antibody Analysis 200 assay on the LabChip GXII system, PerkinElmer) under non-reducing conditions by relative quantification of the expected protein size to total protein content Additional effective titre measurements were performed using quantitative size exclusion liquid chromatography (Dionex UltiMate 3000 UHPLC PREP System & Vanquish Flex UHPLC Systems, Thermo Fisher Scientific Inc.) coupled with mass spectrometry (Q Exactive UHMR Hybrid Quadrupole Orbitrap, Thermo Fisher Scientific Inc.), which will be subsequently denoted as qSEC-MS effective titre. The raw data was further processed with the Chromeleon™ Chromatography Data System (CDS) Software (Thermo Fisher Scientific Inc.). The last three measurements of titre with protein A chromatography, CE-SDS main peak percentage and qSEC-MS effective titre were measured only on supernatant harvested from fed-batch fermentation day 14 (hereafter referred to as Titer Cedex Day 14, Titer Protein A, Eff. Titer SECMS).

### Sample Measurements

### Metabolomics by LC-MS

Quantification of cell culture media components and metabolites in cell culture supernatants were conducted via fully automated ultra-high performance liquid chromatography tandem mass spectrometry method (LC-MS) after a protein precipitation.

### Sample Preparation and Protein Precipitation

Cell culture supernatants were diluted 1:50 with an acetonitrile:methanol, 75:25 (v/v) and 0.2% formic acid, solution. Adding of the sample diluent leads to protein precipitations, which can be removed by subsequent centrifugation at 4500×g for 10 minutes at 4° C. The supernatant was transferred to a 96-well-plate. To improve measurement accuracy, an internal standard of isotopically labelled analytes were added to calibration standards and samples in equal measure.

### Compound Separation

The LC-MS uses a hydrophilic interaction liquid chromatography (HILIC) column (AdvanceBio MS Spent Media Column, Agilent, Santa Clara, USA) for separation of the different analytes in the sample. As mobile phase, acetonitrile (ACN), water solutions with an acidic buffer of 0.1% formic acid and 5 mM ammonium formate were used with a gradient of first 100% ACN:H₂O, 95:5 (v/v) to 50% H₂O:ACN, 60:40 (v/v).

### Quantification and Evaluation

The Ultivo QQQ creates ions via electrospray ionization. Three connected quadrupoles select precursor ions, fragment them and reselect product ions. For evaluation, the MassHunter Quantitative Analysis software (Agilent, Santa Clara, USA) was used to convert the measured peak areas to concentrations by application of a calibration curve, which consisted of up to 10 calibration standards of different concentrations.

### Metabolomics by ICP-MS

Quantification of trace elements in cell culture supernatants was conducted via single quadrupole inductively coupled plasma mass spectrometry (ICP-MS).

### Sample Preparation

Sample dilution and for sample transfer from vials to the ICP-MS the autosampler prepFAST from ESI (ESI Elemental Service & Instruments GmbH, Germany) was used. The elements of interest were separated in 4 different stock solutions. All stock solutions and calibration standards are provided by ESI. For improvement of measurement accuracy, an internal standard is spiked to each sample.

### Trace Elements Ionization

ICP-MS uses an inductively coupled argon plasma to ionize trace elements. The plasma dries aerosol droplets, dissociates the molecules and removes an electron forming single charged ions. The measurements were conducted in the collision energy discrimination mode with hydrogen and helium as reaction gas and collision gas, respectively. The gasses are used to remove double charged ions or dimers.

### Quantification and Evaluation

The separated ions are detected by their mass-to-charge ratio with a quadrupole mass spectrometer. Quantification is done by calculating the concentration of the analyte with a calibration curve, which is generated with the intensity of each compound concerning the internal standard response. Data acquisition and evaluation is performed using Qtegra Intelligent Scientific Data Solution (ISDS) Software (Thermo Scientific).

### Metabolomics by Cedex Bio HT Analyzer

The Cedex Bio HT Analyzer is an automated computerized analyser for determination of analytes in cell culture media. The system enables quantification of substrates, metabolites, electrolytes and antibody titres. In this work, the Cedex Bio HT Analyzer was used for fast measurements of metabolites and for determination of product titre.

### Sample Preparation

300 μL of supernatant were pipetted into single reaction tubes or a multi-well plate, respectively.

### Determination of Product Titre

The Cedex assay to quantify the product titre is based on an immunoturbidimetric method, which uses a rabbit-derived antiserum containing an anti-IgG-antibody. These detection antibodies bind to the constant fragment (Fc) of the produced IgG molecules of interest in the sample, which results in an emerging turbidity due to the rising number of antibody-antigen complexes. Then, absorbance is measured at 340 nm, which is related to the concentration of present IgG (CustomBiotech, 2019).

### RNAseq Measurements

RNA was isolated from the pellets using the Direct-zol RNA MiniPrep Kit from Zymo Research according to the protocol. The library prep was done using a proprietary rRNA depletion protocol. The RNA sequencing was performed on a NovaSeq 6000 System (Illumina, Inc) in S4 flowcell using read mode SE100 for the first 3 projects and PE100 for the remaining 8 projects. Due to low RNA concentration 12 samples were discarded from the sequencing measurements.

### Rapidfire-MS Measurements

For the screening of the therapeutic protein main product and side products, culture supernatants were analysed by high throughput rapidfire-mass spectrometry (RapidFire 365 with QToF 6545 Agilent Technologies Inc). The supernatants were pretreated by removal of cell media and therapeutic protein enrichment. Deconvolution of raw spectra within the elution time window was performed using Byos intact mass workflow by Protein Metrics (Protein Metrics LLC, Cupertino, USA).

### Sample Preparation

Enrichment of the therapeutic protein was performed using different capturing beads depending on the protein profile (e.g.: Toyopearl AF-rProtein A-650F (Tosho), Capture Select KappaXL (Thermo Fisher), PE Purabead 6HF (in-house)). The pretreatment was prepared in 96-well MultiScreen Plate (MAHVN4550, Merck Millipore). In short, after initial washing and equilibration using 100 mM Ammonium Acetate in water, 20 ul of beads were loaded with sample supernatant (up to 4×100 ul, shaking, 5 min). Between each step the plates were stacked as a 2-plate sandwich and centrifuged at 1000 g for 1 min to separate any solution from the beads on the filter plate surface. Afterwards, the samples were washed with 90 mM ammonium acetate in 10% acetonitrile to remove any additional media. The last washing step includes an eluting solution of 39% water, 60% acetonitrile and 1% formic acid to release the therapeutic protein products from the loaded beads.

### Product Measurements

A custom prepared C4 cartridge (4000 A) (Optimize Technologies, USA) was used to trap the proteins at 10 sec at 0.2 ml/min in EluentA (94.9% Water/5% ACN (0.095% FA, 0.005% TFA). The samples were injected into the mass spectrometer for 15 sec at 0.3 ml/min in Eluent B (60% ACN/39.9% Water, 0.095% FA, 0.005% TFA). The QToF mass spectrometer was set in positive ESI-mode with high sensitivity in an extended mass range up to 10 kDa (used mass range from 1300-5000 Da). Additional settings comprised: gas temperature at 350° C., sheet gas temperature at 400° C., drying gas flow at 10 L/min, sheath gas flow at 9 L/min, nebulizer at 60 psi, fragmentor voltage at 410V, skimmer at 130V, capillary voltage at 4500V (VCap) and nozzle voltage at 2000V.

The samples of Project 5 showed many glycosylation patterns in the Fc part. As a result, Protein A capturing of the products was not possible as described in the sample preparation. At the same time the low concentration and volume did not allow sample de-glycosylation. Therefore, rapidFire-MS measurements were not performed for samples of Project 5.

### Data Preprocessing

### Product Titre Variables

The performance of the clones was evaluated after the 14 days fed batch in Ambr15 bioreactors. In this study we have collected various measurements for evaluating the performance of the cell clones during the Ambr15 fermentation (Table 2). We have collected three different titre analytics, from three different technologies, Cedex Bio HT Analyzer, Protein A and qSEC-MS. On days 10, 12, and 14 of the fed batch fermentation (day 10, 12, and 14), titre was measured using Cedex Bio HT Analyzer. Additional titre measurement was performed using analytical protein A chromatography on day 14. On the same day the main product quality was assessed using CE-SDS. CE-SDS measures the amount of correctly assembled main product contained in a concentration of 100 mg/L product. Multiplying the titre measured by Cedex BioHT with the main product percentage abundance (assessed by CE-SDS) we created the measure of effective titre Cedex and similarly the effective titre Protein A. Additionally effective titre was measured directly by a native quantitative SEC-MS (qSEC-MS) chromatography and analysis of the main product amount in harvested cell culture supernatant. Titer Cedex Day 10, Titer Cedex Day 12, Titer Cedex Day 14, Titer Protein A, ME CESDS, Eff. Titer SECMS, Eff. Titer Protein A, Eff. Titer Cedex Day 14 were used as target variables to predict in the machine learning model. The production of three different effective titer measurements was used also to identify problematic samples: samples showing 0 effective titre in one measurement and more than 1000 mg/L in the other were excluded from the following ML analysis.

TABLE 2
Measurements for cell clone performance evaluation.

| Performance | | Fermentation | | Variable |
|---|---|---|---|---|
| Metric | Technology | Day | Unit | annotation |
| Titer | Cedex Bio HT Analyzer | Day 10 | mg/L | Titer Cedex Day 10 |
| Titer | Cedex Bio HT Analyzer | Day 12 | mg/L | Titer Cedex Day 12 |
| Titer | Cedex Bio HT Analyzer | Day 14 | mg/L | Titer Cedex Day 14 |
| Titer | Protein A | Day 14 | mg/L | Titer Protein A |
| Main Product | CE-SDS (peak of main product) | Day 14 | % | MP CESDS |
| Effective Titer | qSEC-MS (peak of main product) | Day 14 | mg/L | Eff. Titer SECMS |
| Effective Titer | CE-SDS*Protein A | Day 14 | mg/L | Eff. Titer Protein A |
| Effective Titer | CE-SDS*Cedex Bio HT Analyzer | Day 14 | mg/L | Eff. Titer Cedex |
| | | | | Day 14 |

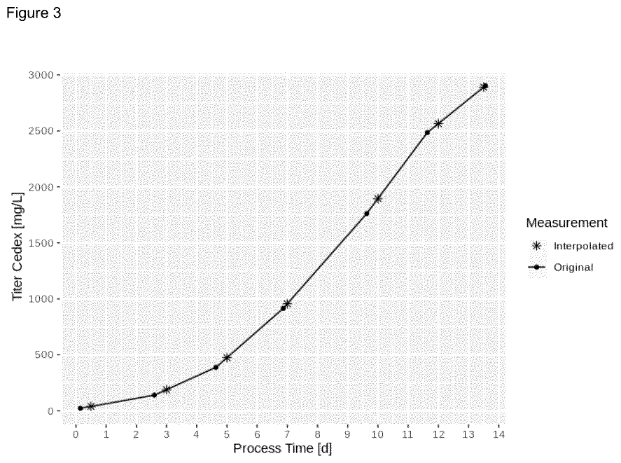

The Cedex Bio HT Analyzer measurements were performed at slightly differing timestamps during the process. For example, measurements of day 10 were taken in a range of 9.5 to 10.5 days, similarly for day 12 in a range of 11.5 to 12.5 days. To harmonize these small differences we used linear models to interpolate titer values at specific timepoints: 0.5, 3, 5, 7, 10, 12 and 13.5 (later mentioned as Day 14) (FIG. 3).

### Standardization of Product Titter Variables

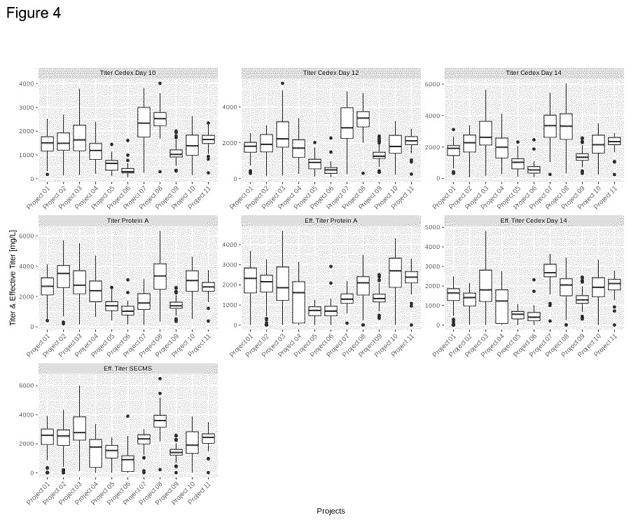

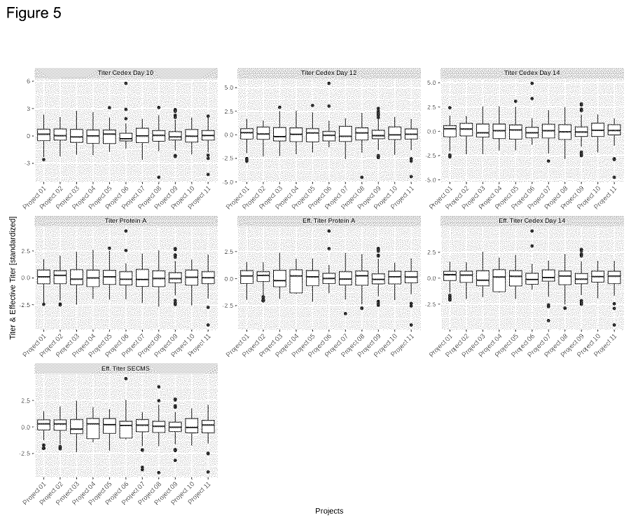

The production rates of cell clones are highly dependent on the complexity of the expressed therapeutic protein. This also holds true for the titre values of the 11 different therapeutic protein formats considered here. FIG. 4 depicts the diversity of the titre ranges produced by the cell clone per project. Some projects' variables are distributed in a narrower range of titre (approx. 0-2000 mg/L), like Projects 05 & 06, others, like Projects 3, 7 & 8 are distributed in a wider range (approx. 0-6000 mg/L). To accommodate for this variability, we applied a per project standardization approach, in which the titre variables were normalized to a standard scale (μ=0 and σ=1) within each project. FIG. 5 shows how the project-by-project standardization approach transforms the variable's differing distribution to a uniform distribution across the 11 different projects. Note worthily, the project-by-project standardization has a biological interpretation on the titre variables. Scaled variables shift the distributions in a way that low-high producer cell clones are aligned across the different projects, irrespectively of the original titre volume produced. For example, as shown in FIG. 4 the titre of the high producers of Project 3 lies between 2000-3500 mg/L, whereas the titre of the high producers of Project 4 is lies in the range from 1500-2500 mg/L. However in FIG. 5, these two different ranges are equalized. Meanwhile extreme producer clones (with very high or low titre) per project are not influenced. Since the titre variable “MP CESDS” already represents a percentage of main product per clone, it is already scaled in clonal level.

### RNAseq Data Preprocessing

The quality of the raw fastq files was evaluated using the FastQC software (version 0.11.9). Adapter sequences (ILLUMINACLIP:TruSeq3-PE.fa:2:30:10) were removed by Trimmomatic software (version 0.39). Sequences for the transgene and the reference genome were mapped and further analysed, separately. Reads were aligned to the reference genome file (CriGri-PICR, RefSeq Assembly accession: GCF_003668045.1) [41] using the HISAT2 software (version 2.2.1). Feature quantification was performed using HTseq (version 0.13.5). Both reference genome and genome annotation files were retrieved from the NCBI database (Release 252: Oct. 15, 2022). The final reads were normalized to the library size and library composition using DESeq2's (version 1.38.3) [42] median of ratios.

The transfected cassette genes used as input features for the machine learning part consist of the heavy chains, light chains, the 5′ UTR regions and the gene expressing resistance to the selection agent. Some of the therapeutic proteins consist of one heavy (HC) and light chain (LC), others of two heavy (HC1 and HC2) and light chains (LC1 and LC2) and others only of one chain (C). The reads from the transfected cassette genes were normalized to the gene size. To ensure that all the projects are having the same set of chains, we transformed the features and merged them in one homogeneous set across the different projects. In particular, for the formats missing the respective chain features we applied the following calculations: HC=HC1+HC2, LC=LC1+LC2, C=HC+LC, HC1=HC/2, HC2=HC/2, LC1=LC/2, LC2=LC/2, & HC1=C/4, HC2=C/4, LC1=C/4, LC2=C/4. To further enrich the feature space we included the ratios of expressed sequences as follows: HC/LC, LC1/LC2, HC1/HC2, HC1/LC1, HC1/LC2, HC2/LC1, HC2/LC2, C/LC, C/HC, C/HC1, C/HC2, C/LC1, C/LC2, C/5′ UTR.

### Metabolomics Data Preprocessing

The final concentrations of the metabolites screened by LC-MS, ICP-MS and Cedex Bio HT Analyzer were converted to cell-specific concentrations by the following procedure. During the supernatant harvest day, we measured the viable cell density and the average cell volume of each culture sample. We calculated the cell-specific concentrations by dividing the concentration of each metabolite by the viable cell density and the average cell volume. Additionally while cleaning and preparing the data, missing values of the metabolites were imputed to the mean metabolite value.

### Rapidfire-MS Data Preprocessing

The final mass-to-intensity data generated after the m/z deconvolution were further processed using a Gaussian Mixture Model (GMM) [43] approach. The GMM was fitted via Expectation Maximization (EM) [44] algorithm. The purpose of this was to align the main and side products produced within and across the several projects. The homogenized feature space produced is then suitable for the following machine learning pipeline.

### Random Forest Regression

We used machine learning regression to predict the continuous values of each product titre variable, namely titre, main product and effective titre. Regression analysis is a method for investigating functional relationships between input features and target variables [45]. Here input features are the multiple omics data and the target variables are the titre, main product and effective titre. We applied a random forest regression model [46] to evaluate the predictive validity of the early stage omics data and identify features with high correlation to the cell clones titre variables. Random forests consist of multiple random decision trees. Decision Trees are great for obtaining non-linear relationships between input features and the target variable [47]. It is an ensemble of decision trees, meaning many trees are constructed in a certain “random” way. Each tree is created from a different sample of observations and at each node, a different sample of features is selected for splitting. Each of the trees makes its own individual prediction and these predictions are then averaged to produce a single result.

For the implementation of the machine learning algorithm, we used the packages caret (version 6.0-92, [48]) and randomForest (version 4.7-1, [49]) from R CRAN. Every model was set to grow 1000 trees (ntree=1000) and the number of variables randomly sampled as candidates at each split was set to a random search of 15 different combinations of variables (tuneLength=15).

### Train-Validation-Test Split

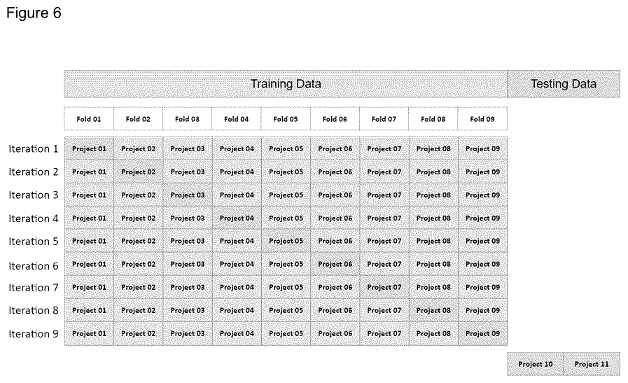

We used the K-fold cross-validation hold-out method for hyperparameter tuning. K-fold cross validation uses part of the available data to fit the model, and a different part to test it [50]. This process is repeated K times with different random partitioning to generate an average performance measure from K models. In this study, we performed stratified partitioning using in each fold the data generated from one set of samples from the same therapeutic protein as validation set and as training set the data generated from the rest of the projects. In that way, we achieved a format-fold cross validation setup in which for each model training-validation iteration the data from one project is left out and the model is trained on the data from the rest of the projects. Eventually, the model tries to predict the values of the left out project, without having been trained on any data from its own samples. This process is repeated until all projects have been predicted once by the model. As shown in FIG. 6, for this per project-fold cross validation approach we used the data from the first 9 projects (Projects 1-9). Projects 10 and 11 were used as testing sets, after the model was trained. Since projects 10 and 11 are completely unknown to the model they provide an unbiased evaluation of the final model fit. This setup excludes information leakage between training and testing set and at the same time mimics a real case scenario, where our model should predict unseen data from new therapeutic proteins.

### Baseline Model Comparison

To evaluate the performance of the ML models we compared it to the status quo, which in the early stage of monoclonal cell lines consists of ELISA immunoassay to estimate therapeutic protein titre. Based on this, we select the top hundreds of clones to move forward to the CLD pipeline. To build the baseline model, we used the early stage titre ELISA measurements as input features in a single variable linear regression model to correlate to the same product titre variables (target variables) we have predicted with the ML models. We applied this comparison project by project. In this way, we obtained a baseline coefficient of determination (R²) per project, which gives a metric of the proportion of variation in the product titre variable that is explained by the early stage titre ELISA measurements. Through the comparison of the baseline R² to the model prediction R², we can ascertain whether our omics assays' model performs better or worse compared to the status quo. While the early stage ELISA measurement provides a reasonable representation of the titre of the therapeutic protein, it is not providing a metric for the quality of the product in that stage. To establish a product quality baseline model, we used the main product measured by the rapidFire-MS assay described earlier. In this case, we created a linear regression model using the rapidFire MS main product values as input variable and the main product measured by CESDS in the later stage as output variable. Similarly, for the evaluation of the models predicting the later stage effective titre variables we used a combination of the early stage titre ELISA measurements with the main product measured by the rapidFire-MS to create an early stage effective titre variable. Respectively, we used the latter to correlate to the late stage effective titre variables, obtain the R2 and compare to the omics assays' model ability to predict the late stage effective titre. Table 3, presents the input and output variables of each baseline model constructed for each product titre variable predicted.

TABLE 3
Components of the baselines models contracted
per product titer variable.

| | Product titer variables |
|---|---|
| Baseline metric (Input Variables) | (Output Variables) |
| Titer ELISA measurements | Titer Cedex Day 10 |
| Titer ELISA measurements | Titer Cedex Day 12 |
| Titer ELISA measurements | Titer Cedex Day 14 |
| Titer ELISA measurements | Titer Protein A |
| Main Product rapidFire-MS | MP CESDS |
| Titer ELISA measurements * MP | Eff. Titer SECMS |
| rapidFire-MS | |
| Titer ELISA measurements * MP | Eff. Titer Protein A |
| rapidFire-MS | |
| Titer ELISA measurements * MP | Eff. Titer Cedex Day 14 |
| rapidFire-MS | |

### Models Comparison Metrics

We have created a model for each omics assay (metabolomics, transcriptomics and rapidFire-MS) and 4 multi assay models with the combinations of the individual assays. Each model is evaluated according to the model's coefficient of determination R² compared to the baseline R². To assess the predictivity of each assay separately, we used the data from Projects 1-9 in the per project cross validation setup as described earlier. As an indicator of the difference between the R² populations between baseline and RF model we used the non-parametric Wilcoxon test [51]. We performed a one-tailed paired test, testing whether the mean difference is less than 0 [52]. In this case, the null hypothesis (Ho) corresponds to the mean difference (Baseline R²−Assay Model R²)>=0 and the alternative hypothesis (Ha) to the mean difference (Baseline R²−Assay Model R²)<0. When the outcome of adjusted p-value (p-value adjustment method: Bonferroni correction) is less than the significance level alpha=0.05, we can reject the null hypothesis and conclude that the mean R² population is significantly higher in the assay model predictions than the baseline model. The results of each model are examined later, separated in single and multi assay models.

### Results

### Single Assay Models

### Metabolomics

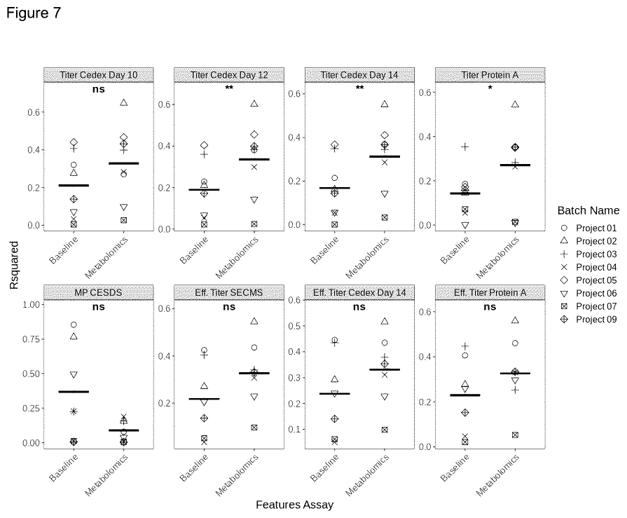

For the training of metabolomics model, Projects 1-7 & 9 were used. Project 8 was excluded due to inability to acquire the relevant data. FIG. 7, shows the per project prediction of the multi-feature metabolomics model compared to the baseline prediction for each product titre variable. In the case where the model predicts titre and effective titre SEC-MS, the average prediction rate of the metabolomics model is significantly higher than the baseline. On the contrary the metabolomics set is not predictive for the product quality attribute. The metabolomics features that are identified from the model as the most informative for the prediction are: IgG, Formate, Pyridoxamine, Asymmetric dimethylarginine, Methionine Sulfoxide, Alanin, Lactic acid, Ethanolamine, Pyruvic acid, Acetate, Glycine, Isoleucine, Tin and Vanadium.

### RapidFire-MS

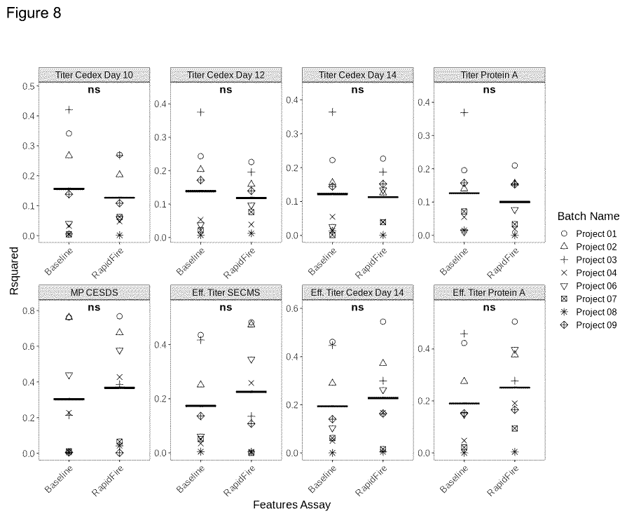

For the training of the RapidFire model Projects 1-4 & 6-9 were used. Project 5 was excluded due to inability to acquire the relevant data (see section RapidFire-MS Measurements). FIG. 8, shows the per project prediction of the multi-feature RapidFire-MS model compared to the baseline prediction for each product titre variable. We observe that the average rapidFire-MS model prediction rate is higher than the baseline in the case that the model is predicting product quality variables, the main product from CESDS and the effective titre variables. According to the paired Wilcoxon test, this increase is not statistically significant. On the contrary, the rapidFire-MS set is not showing any increased predictive rate for the variables that do not include product quality attribute. An additional point to be mentioned here is that the comparison of the baseline versus rapidFire-MS for MP CESD and effective titre variables is a comparison between the main peak identified by the rapidFire-MS versus the feature sets of all the peaks identified. This indicates that, additionally to the main peak, the other peaks annotating several side products add to the increased predictive capability of the RapidFire model. The model identifies the majority of the features important, with top the main product peak (100) and the side product peaks of molecular weight 82%, 78%, 56%, 50%, 38%, 34%, 32%, 28%, 16% of the main product molecular weight.

### Transcriptomics

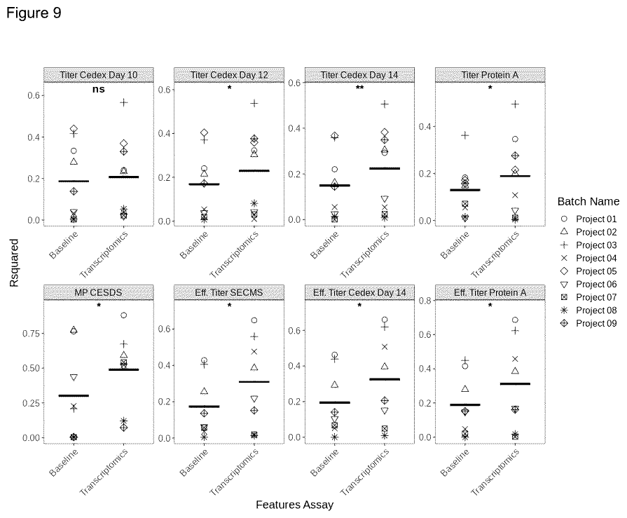

For the training of the transcriptomics measurements Projects 1-9 were used. FIG. 9, shows the per project prediction of the multi-feature transcriptomics model compared to the baseline prediction for each product titre variable. The average prediction rate of the transcriptomics model is statistically significant higher than the baseline for almost all the product titre variables The 10 most informative features are identified the ratios of C/HC hole, C/LC1, LC1/LC2, HC1, HC2, HC1/LC1, HC2/LC1, HC1/LC2, C/HC2 & LC1.

### Multi Assay Models

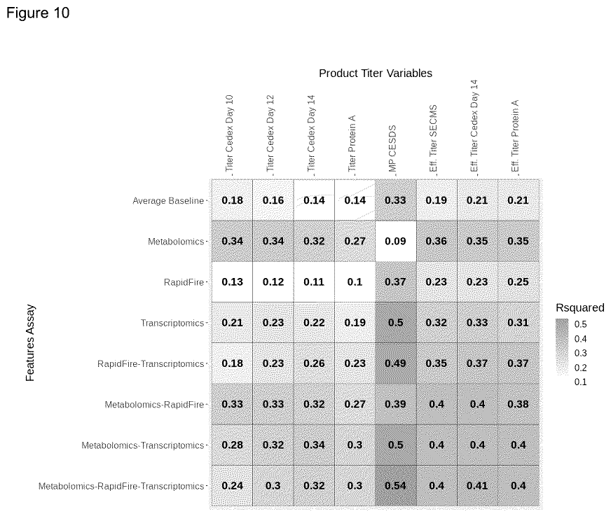

We evaluated the predictive capability of four different multi assay models. The models are combining the features of a) Metabolomics and RapidFire-MS, b) Metabolomics and Transcriptomics, c) RapidFire-MS and Transcriptomics and d) Metabolomics, Rapidfire-MS and Transcriptomics. In that way we want to assess how combining the features of the different assays can offer a higher predictivity compared to the single assay models. FIG. 10, demonstrates a heatmap of all the average R² generated from each baseline, single and multi assay model. We observe that the Metabolomics model itself can predict the first four product titre variables as well as the multi assay models, with an increase of 0.17 from the baseline model. Whereas, the same model is not predicting adequately the MP CESDS quality attribute. RapidFire-MS and Transcriptomics are performing better in predicting the product quality target variable with a 0.16 increase from the baseline prediction. However not as good when predicting titre. This indicates why the multi assay models are capable of a higher rate of prediction of the effective title variables. Performance indicators combining titre and product quality target variables, such as effective titre, are supporting the endpoint decision making during monoclonal cell line selection in the early stage of CLD. Thus the prediction rates of the multi assay models are indicating that a multi-omics machine learning model is superior in predicting the proportion of variation of the therapeutic protein production attributes during the later performed production process in fed-batch bioreactors, compared to the status quo assay. Indicatively the final model testing using the last two unseen Projects 10 and 11 will be examined in the near future, offering additional data.

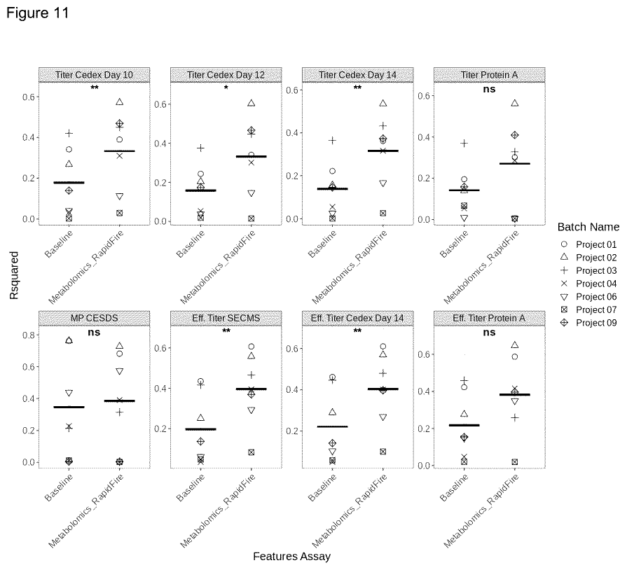

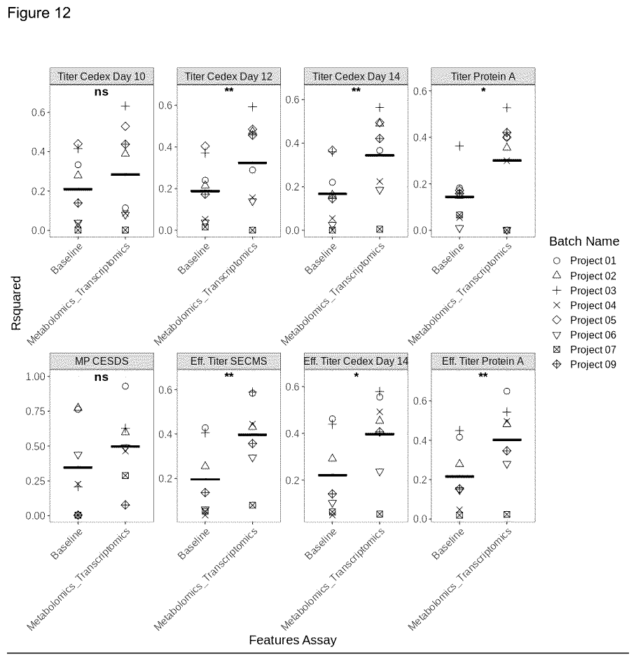

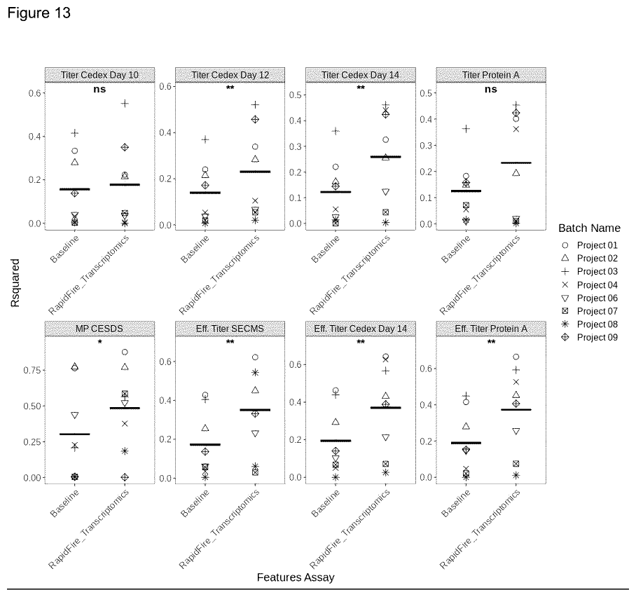

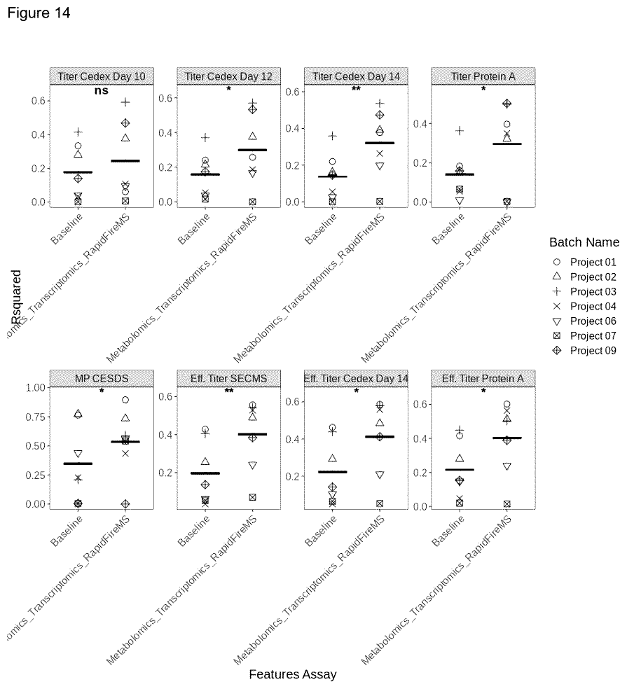

In FIGS. 11-14, we can see a more detailed demonstration behind each multi assay model, similarly to those demonstrating the single assay models. In all the models consisting of more than one assay the prediction rate of the assays model is significantly higher compared to the baseline model, for the majority of the product titre variables. In addition, the models are adequately predictive for the majority of projects and the molecule formats examined in this study, as most of the projects show predictivity higher than the average baseline. Particularly the multi assay models, show the higher predictive rates with an average 0.2 increase from the baseline. The multi assay model comprising metabolomics, transcriptomics and rapidFire-MS input features, performs better than the rest with an average 0.16 increase in the R² compared to the baseline. However, a lack of predictability is noticed for Projects 7 and 8 that have dutaFab or no antibody format. Both single assay and multi assay models seem to have low predictive power on these projects. This could be due to the fact that our dataset is not rich with data coming from this kind of therapeutic protein indicating that enrichment of the model would expand its predictive capabilities.

### DISCUSSION

This study represents one of the largest and most diverse studies of CHO multi omics datasets published to date. Data from 892 different monoclonal cell lines, covering 11 different difficult to express therapeutic proteins, were generated in the early phase of cell clones during CLD. The early phase omics data library is used to build ML models capable of predicting antibody production attributes of the later performed production process in fed-batch bioreactors. We evaluated the predictive capability of each individual omics assay as well as the several combinations of them using random forest regression models, set in a rigorous training-validation-testing approach.

In total, 7 different omics models are constructed, each one consisting of the following omics features i) metabolomics, ii) rapidFire-MS, iii) transcriptomics, iv) metabolomics and rapidFire-MS, v) metabolomics and transcriptomics, vi) rapidFire-MS and transcriptomics and vii) metabolomics, rapidfire-MS and transcriptomics. Each feature set is used to predict 8 different product titre variables, which have been measured after incubation in fed-batch bioreactors systems. To assess how well the observed product titre variables are replicated by each omics features set, we compared each model's coefficient of determination R² produced in a project-by-project prediction setup to a baseline model R². The single omics assay models demonstrate to be predictive in different product titre variables. For titre attributes the average prediction rate of the metabolomics model is significantly higher than the baseline. Whereas, product quality and effective titre attributes are predicted better with the RapidFire-MS model. The transcriptomics model shows significant higher performance for the majority of the product variables predicted. Additionally, the models consisting of more than one omics assay feature set, demonstrate a significantly higher prediction rate than the baseline model and increased prediction rate compared to the single omics assay models. For example, the prediction of the effective titre variable measured by SEC-MS by the baseline model results in a R²=0.19, by the single assay models increases to approximately 0.36 and up to 0.4 by the multi assay models. More detailed results are shown in FIG. 10. This indicates that the multi assay input features adds value to the predictive capability. In other words, the variation in the cell clones performance observed after the later stage fed-batch bioreactors systems can be better predicted by a combination of several different omics features, than using features generated from a single assay alone.

From an industrial point of view, an ML model able to predict the future performance of the dynamic system of the clones can substantially improve the current lab screening workflows. After single cell cloning, clonal expansion and evaluation typically consists of hundreds of cell clones and requires months until a dozen candidates are selected as the highest producers for up-scaling [7]. The model proposed here, can offer the outcome of the highest producer clones much earlier. A proposed approach would be to measure the omics feature set during the early phase of clonal recovery after single cell cloning and feed them to the model. Then, the clones can be ranked based on the prediction of titre attribute as given by the model. In short, the clones predicted as the highest performer can proceed directly to the up-scaling phase, while omitting the extensive expansion and characterization steps. A future aspect of this approach would be to set the model in a continuous improvement circle of active learning [53]. Over time, the model can learn from new experimental data, improving its predictions and recommendations for future cell line development efforts. In that way, the model will be able to identify highly productive cell clones with greater accuracy. Enrichment of the training dataset will improve the predictivity of the model. It will be able to predict a wider variety of different format molecules making it applicable in broader case scenarios of therapeutic proteins.

The ML models disclosed herein can aid in the selection of the most suitable cell clones for therapeutic protein production, offering the benefits of a faster and less exhaustive experimental effort. At the same time benchmarking of an automated ML method in a lab pipeline offers process transparency, increased process robustness and rational data-driven decisions. Being able to predict from the early phase of cell clones which ones will end up being high producers, can result in saving approximately four months in the whole CLD workflow by selecting and furtherly scaling up only the top clones as predicted by the model. Correspondingly, the whole cell line and bioprocess development pipeline of the “difficult to express” recombinant proteins will be substantially improved.

### Example 2

The goal of this work was to develop a screening system that allows the selection of highly productive cell clones for mAb production. This selection should be made possible early in the cell line development process and consequently would require substantially less resources to achieve the same or better success rate as current methods. This will enable selecting a small number of cell clones by using the machine learning disclosed herein on the data generated in multi-well plates (primary screening of cell clones after single cell cloning), and go straight to a lab-scale bioreactor evaluation stage (Ambr250). This will not only save weeks of effort and material, will also have a better chance of producing highly productive cell lines compared to the standard approach.

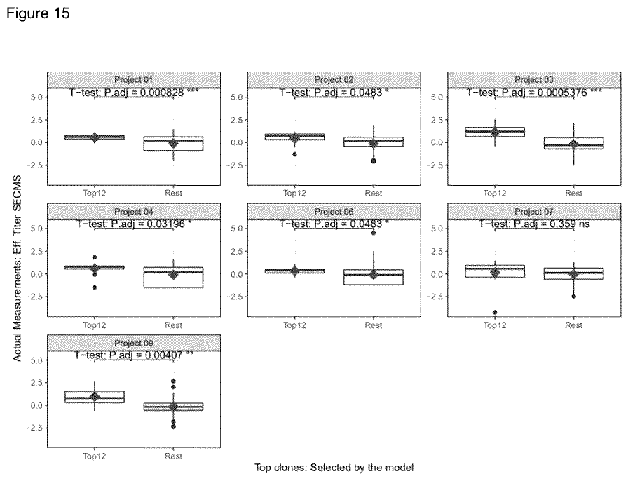

To evaluate if the model can predict the top 12 clones adequately, the top 12 clones predicted by the model were compared to their actual effective titre measurements. In particular, we used the predictive values for effective titre measured by qSEC-MS, as they are given by the multi assay model consisting of metabolomics, rapidFire-MS and transcriptomics. We sorted the cell clones according to the predicted values and selected the top 12 clones with the highest predicted variable. Then we compared the mean population of the actual Eff. titre measured by qSEC-MS of the top 12 clones to the rest of the clones. FIGS. 15 & 16, show the boxplots of the two populations “top12” and “rest” as predicted by the model per project. We performed a t-test comparing the mean of the two populations. For all the projects apart from Project 7, the population of the top 12 clones has a significantly higher mean of titre compared to the rest of the population. This means that the clones selected by the models as the highest producers are actually high producers and they fall above the average of the distribution of the titre.

Based on these additional data we propose the following approach: First the omics input features need to be measured during the early phase of clonal recovery after single cell cloning. The new measurements are used as input to the model, which in turn predicts a titre. The cell clones can be ranked based on this prediction and the top 12 clones with the highest predicted effective titre proceed directly to the Amb250 fermentation. In this way, both time and resources are saved, since there is no need for extensive scale up.

### REFERENCES

All references cited herein are incorporated herein by reference in their entirety and for all purposes to the same extent as if each individual publication or patent or patent application was specifically and individually indicated to be incorporated by reference in its entirety. The specific embodiments described herein are offered by way of example, not by way of limitation. Any sub-titles herein are included for convenience only, and are not to be construed as limiting the disclosure in any way.
1. Walsh, G. (2018) Biopharmaceutical benchmarks 2018. Nat Biotechnol 36: 1136-1145.
2. Mullard, A. (2021) FDA approves 100th monoclonal antibody product. Nat Rev Drug Discov 20: 491-495.
3. Tihanyi, B., and L. Nyitray (2021) Recent advances in CHO cell line development for recombinant protein production. Drug Discov Today Technologies 38: 25-34.
4. Lai, T., Y. Yang, and S. K. Ng (2013) Advances in Mammalian Cell Line Development Technologies for Recombinant Protein Production. Pharm 6: 579-603.
5. Bolisetty, P., G. Tremml, S. Xu, and A. Khetan (2020) Enabling speed to clinic for monoclonal antibody programs using a pool of clones for IND-enabling toxicity studies. Mabs 12:1763727.
6. Gross, A., J. Schoendube, S. Zimmermann, M. Steeb, R. Zengerle, and P. Koltay (2015) Technologies for Single-Cell Isolation. Int J Mol Sci 16: 16897-16919.
7. Castan, A., P. Schulz, T. Wenger, and S. Fischer (2018) Cell Line Development. pp. 131-146. In: Jagschies, G., Lindskog, E., tacki, K., and Galliher, P. (eds.). Biopharmaceutical processing: development, design, and implementation of manufacturing processes. Elsevier.
8. Ko, P., S. Misaghi, Z. Hu, D. Zhan, J. Tsukuda, M. Yim, M. Sanford, D. Shaw, M. Shiratori, B. Snedecor, M. Laird, and A. Shen (2018) Probing the importance of clonality: Single cell subcloning of clonally derived CHO cell lines yields widely diverse clones differing in growth, productivity, and product quality. Biotechnol Progr 34: 624-634.
9. Hsu, W.-T., R. P. S. Aulakh, D. L. Traul, and I. H. Yuk (2012) Advanced microscale bioreactor system: a representative scale-down model for bench-top bioreactors. Cytotechnology 64: 667-678.
10. Moses, S., M. Manahan, A. Ambrogelly, and W. L. W. Ling (2012) Assessment of AMBR™ as a model for high-throughput cell culture process development strategy. Adv Biosci Biotechnology 2012: 918-927.
11. Li, F., N. Vijayasankaran, A. (Yijuan) Shen, R. Kiss, and A. Amanullah (2010) Cell culture processes for monoclonal antibody production. Mabs 2: 466-479.
12. Yang, W., J. Zhang, Y. Xiao, W. Li, and T. Wang (2022) Screening Strategies for High-Yield Chinese Hamster Ovary Cell Clones. Frontiers Bioeng Biotechnology 10: 858478.
13. Rameez, S., S. S. Mostafa, C. Miller, and A. A. Shukla (2014) High-throughput miniaturized bioreactors for cell culture process development: Reproducibility, scalability, and control. Biotechnol Progr 30: 718-727.
14. Zhu, M. M., M. Mollet, R. S. Hubert, Y. S. Kyung, and G. G. Zhang (2017) Handbook of Industrial Chemistry and Biotechnology. Handb Industrial Chem Biotechnology DOI: 10.1007/978-3-319-52287-6_29.
15. Chen, Y.-J., M. Chen, Y.-C. Hsieh, Y.-C. Su, C.-H. Wang, C.-M. Cheng, A.-P. Kao, K.-H. Wang, J.-J. Cheng, and K.-H. Chuang (2018) Development of a highly sensitive enzyme-linked immunosorbent assay (ELISA) through use of poly-protein G-expressing cell-based microplates. Sci Rep-uk 8: 17868.
16. Sawyer, W. S., N. Srikumar, J. Carver, P. Y. Chu, A. Shen, A. Xu, A. J. Williams, C. Spiess, C. Wu, Y. Liu, and J. C. Tran (2020) High-throughput antibody screening from complex matrices using intact protein electrospray mass spectrometry. Proc National Acad Sci 117: 9851-9856.
17. Tejwani, V., M. Chaudhari, T. Rai, and S. T. Sharfstein (2021) High-throughput and automation advances for accelerating single-cell cloning, monoclonality and early phase clone screening steps in mammalian cell line development for biologics production. Biotechnol Progr 37: e3208.
18. Bauer, N., B. Oswald, M. Eiche, L. Schiller, E. Langguth, C. Schantz, A. Osterlehner, A. Shen, S. Misaghi, J. Stingele, and S. Auslander (2022) An arrayed CRISPR screen reveals Myc depletion to increase productivity of difficult-to-express complex antibodies in CHO cells. Synthetic Biology 7: ysac026.
19. Huang, Z., and S. Yoon (2020) Identifying metabolic features and engineering targets for productivity improvement in CHO cells by integrated transcriptomics and genome-scale metabolic model. Biochem Eng J 159: 107624.
20. Karottki, K J. la C., H. Hefzi, S. Li, L. E. Pedersen, P. N. Spahn, C. Joshi, D. Ruckerbauer, J. A. H. Bort, A. Thomas, J. S. Lee, N. Borth, G. M. Lee, H. F. Kildegaard, and N. E. Lewis (2021) A metabolic CRISPR-Cas9 screen in Chinese hamster ovary cells identifies glutamine-sensitive genes. Metab Eng 66: 114-122.
21. Weinguny, M., P. Eisenhut, G. Klanert, N. Virgolini, N. Marx, A. Jonsson, D. Ivansson, A. Lovgren, and N. Borth (2020) Random epigenetic modulation of CHO cells by repeated knockdown of DNA methyltransferases increases population diversity and enables sorting of cells with higher production capacities. Biotechnol. Bioeng. 117: 3435-3447.
22. Masson, H. O., K. J. la C. Karottki, J. Tat, H. Hefzi, and N. E. Lewis (2023) From Observational to Actionable: Rethinking Omics in Biologics Production. DOI: 10.20944/preprints202302.0037.v1.
23. Kolluri, S., J. Lin, R. Liu, Y. Zhang, and W. Zhang (2022) Machine Learning and Artificial Intelligence in Pharmaceutical Research and Development: a Review. Aaps J 24: 19.
24. Helleckes, L. M., J. Hemmerich, W. Wiechert, E. von Lieres, and A. Granberger (2022) Machine learning in bioprocess development: from promise to practice. Trends Biotechnol DOI: 10.1016/j.tibtech.2022.10.010.
25. Narayanan, H., M. F. Luna, M. Stosch, M. N. C. Bournazou, G. Polotti, M. Morbidelli, A. Butté, and M. Sokolov (2020) Bioprocessing in the Digital Age: The Role of Process Models. Biotechnol J 15:1900172.
26. Walsh, I., M. Myint, T. Nguyen-Khuong, Y. S. Ho, S. K. Ng, and M. Lakshmanan (2022) Harnessing the potential of machine learning for advancing “Quality by Design” in biomanufacturing. Mabs 14: 2013593.
27. Povey, J. F., C. J. O'Malley, T. Root, E. B. Martin, G. A. Montague, M. Feary, C. Trim, D. A. Lang, R. Alldread, A. J. Racher, and C. M. Smales (2014) Rapid high-throughput characterisation, classification and selection of recombinant mammalian cell line phenotypes using intact cell MALDI-ToF mass spectrometry fingerprinting and PLS-DA modelling. J. Biotechnol. 184: 84-93.
28. Clarke, C., P. Doolan, N. Barron, P. Meleady, F. O'Sullivan, P. Gammell, M. Melville, M. Leonard, and M. Clynes (2011) Predicting cell-specific productivity from CHO gene expression. J. Biotechnol. 151: 159-165.
29. Barberi, G., A. Benedetti, P. Diaz-Fernandez, D. C. Sévin, J. Vappiani, G. Finka, F. Bezzo, M. Barolo, and P. Facco (2022) Integrating metabolome dynamics and process data to guide cell line selection in biopharmaceutical process development Metab Eng 72: 353-364.
30. Barbed, G., A. Benedetti, P. Diaz-Fernandez, G. Finka, F. Bezzo, M. Barolo, and P. Facco (2021) Anticipated cell lines selection in bioprocess scale-up through machine learning on metabolomics dynamics. Ifac-papersonline 54: 85-90.
31. Kroll, P., A. Hofer, S. Ulonska, J. Kager, and C. Herwig (2017) Model-Based Methods in the Biopharmaceutical Process Lifecycle. Pharmaceut Res 34: 2596-2613.
32. Kotidis, P., and C. Kontoravdi (2020) Harnessing the potential of artificial neural networks for predicting protein glycosylation. Metabolic Eng Commun 10: e00131.
33. Severson, K, J. G. VanAntwerp, V. Natarajan, C. Antoniou, J. Thommes, and R. D. Braatz (2015) Elastic net with Monte Carlo sampling for data-based modeling in biopharmaceutical manufacturing facilities. Comput Chem Eng 80: 30-36.
34. Sarker, I. H. (2021) Machine Learning: Algorithms, Real-World Applications and Research Directions. Sn Comput Sci 2: 160.
35. James, G., D. Witten, T. Hastie, and R. Tibshirani (2013) An Introduction to Statistical Learning, with Applications in R. Springer Texts Stat. DOI: 10.1007/978-1-4614-7138-7_7.
36. Sokolov, M., J. Ritscher, N. MacKinnon, J. Souquet, H. Broly, M. Morbidelli, and A. Butté (2017) Enhanced process understanding and multivariate prediction of the relationship between cell culture process and monoclonal antibody quality. Biotechnol Progr 33: 1368-1380.
37. Narayanan, H., M. Sokolov, M. Morbidelli, and A. Butté (2019) A new generation of predictive models: The added value of hybrid models for manufacturing processes of therapeutic proteins. Biotechnol. Bioeng. 116: 2540-2549.
38. Zürcher, P., M. Sokolov, D. Brühlmann, R. Ducommun, M. Stettler, J. Souquet, M. Jordan, H. Broly, M. Morbidelli, and A. Butté (2020) Cell culture process metabolomics together with multivariate data analysis tools opens new routes for bioprocess development and glycosylation prediction. Biotechnol Progr 36: e3012.
39. Ng, D., M. Zhou, D. Zhan, S. Yip, P. Ko, M. Yim, Z. Modrusan, J. Joly, B. Snedecor, M. W. Laird, and A. Shen (2021) Development of a targeted integration Chinese hamster ovary host directly targeting either one or two vectors simultaneously to a single locus using the Cre/Lox recombinase-mediated cassette exchange system. Biotechnol. Prog. 37: e3140.
40. Carver, J., D. Ng, M. Zhou, P. Ko, D. Zhan, M. Yim, D. Shaw, B. Snedecor, M. W. Laird, S. Lang, A Shen, and Z. Hu (2020) Maximizing antibody production in a targeted integration host by optimization of subunit gene dosage and position. Biotechnol Progr 36: e2967.
41. Rupp, O., M. L. MacDonald, S. Li, H. Dhiman, S. Poison, S. Griep, K. Heffner, I. Hernandez, K. Brinkrolf, V. Jadhav, M. Samoudi, H. Hao, B. Kingham, A. Goesmann, M. J. Betenbaugh, N. E. Lewis, N. Borth, and K. H. Lee (2018) A reference genome of the Chinese hamster based on a hybrid assembly strategy. Biotechnol. Bioeng. 115: 2087-2100.
42. Love, M. I., W. Huber, and S. Anders (2014) Moderated estimation of fold change and dispersion for RNA-seq data with DESeq2. Genome Biol. 15: 550.
43. Reynolds, D. (2009) Gaussian Mixture Models. Encyclopedia of Biometrics DOI: 10.1007/978-0-387-73003-5_773.
44. Moon, T. K. (1996) The expectation-maximization algorithm. IEEE Signal Process. Mag. 13: 47-60.
45. Chatterjee, S., and A S. Hadi (1938) Regression Analysis by Example. Wiley Ser. Probab. Stat. DOI: 10.1002/0470055464.
46. Ho, T. K. (1995) Random Decision Forests. Proceedings of 3rd international conference on document analysis and recognition.
47. Kuhn, M., and K Johnson (2013) Applied Predictive Modeling. Springer DOI: 10.1007/978-1-4614-6849-3.
48. Kuhn, M. (2008) Building Predictive Models in R Using the caret Package. Journal of Statistical Software.
49. Liaw, A, and M. Wiener (2002) Classification and Regression by Random Forest. R News.
50. Hastie, T., R. Tibshirani, and J. Friedman (2017) The Elements of Statistical Learning. Springer.
51. Wilcoxon, F. (1945) Individual Comparisons by Ranking Methods. Biometrics Bulletin.
52. Mann, H. B., and D. R. Whitney (1947) On a Test of Whether one of Two Random Variables is Stochastically Larger than the Other. The Annals of Mathematical Statistics.
53. Bonwell, C., and J. Eison (1991) Active learning: Creating excitement in the classroom. AEHE-ERIC Higher Education Report No. 1. ED340272. Washington, DC: Jossey-Bass.

1. A computer-implemented method for facilitating selection of a cell line, from among a plurality of candidate cell lines that produce a recombinant protein, the method comprising:
(a) receiving omics data for each of the plurality of candidate cell lines; and
(b) using a machine learning model to predict one or more values indicative of recombinant protein titre and/or quality,
wherein the machine learning model has been trained using a training dataset comprising omics data for a multiplicity of cell lines, and for each cell line, one or values indicative of recombinant protein titre and/or quality, and
wherein the omics data comprises:
i) transcriptomics data;
ii) metabolomics data;
iii) proteome data;
iv) transcriptomics and metabolomics data;
v) transcriptomics and proteome data;
vi) metabolomics and proteome data; or
vii) transcriptomics, metabolomics, and proteome data.

2. The method of claim 1, wherein the proteome data comprises data indicative of the titre of the main product and side products in the cell culture supernatant of the respective cell line.

3. The method of claim 1, wherein the omics data corresponding to each cell line in the training dataset is obtained in cell line development process at least one week, e.g. at least two, three, four, or five weeks, of cell culturing, before obtaining values indicative of recombinant protein titre and/or quality.

4. The method of claim 1, wherein the values indicative of recombinant protein titre and/or quality are obtained during small-scale fermentation.

5. The method of claim 1, wherein the omics data of each cell line is obtained at the same time, e.g. from one common cell pellet or cell culture supernatant sample.

6. The method of claim 1, wherein the metabolomics data comprise values corresponding to the concentration of C-nutrient source, N-nutrient source, anions, cations, recombinant protein (e.g. IgG) product, organic acids, total protein, amino acids, amino acid derivatives, vitamins, vitaminoids, metabolic breakdown products, organic acids, amines, formate, pyridoxamine, asymmetric dimethylarginine, methionine sulfoxide, alanin, lactic acid, ethanolamine, pyruvic acid, acetate, glycine, isoleucine, Tin and Vanadium, and/or other chemical elements, in cell culture supernatant.

7. The method of claim 1, wherein the metabolomics data is obtained by one or methods selected from a group consisting of: i) ultra-high performance liquid chromatography tandem mass spectrometry method (LC-MS), preferably after protein precipitation; ii) single quadrupole inductively coupled plasma mass spectrometry (ICP-MS); and iii) Cedex Bio HT Analyzer.

8. The method of claim 7, wherein LC-MS is used for measuring the concentration of cell culture media components and metabolites, and/or ICP-MS is used for measuring the concentration of trace elements in cell culture supernatants.

9. The method of claim 1, wherein the metabolomics data is preprocessed by dividing the values, e.g. the concentration of each metabolite, by the viable cell density and the average cell volume of the corresponding cell culture at the time of harvesting.

10. The method of claim 1, wherein the proteome data is obtained via mass spectrometry, e.g. high throughput RapidFire-mass spectrometry.

11. The method of claim 2, wherein before obtaining the proteome data the supernatants are pre-treated by removal of cell media and recombinant protein enrichment.

12. The method of claim 1, wherein the transcriptomics data is obtained from a cell pellet, preferably by a high-throughput method, e.g. RNA-seq.

13. The method of claim 1, wherein the one or more values indicative of recombinant protein titre and/or quality comprises the recombinant protein titre measured on day 10 (±half day), day 12 (±half day), and/or day 14 (±half day) of the fed batch culture.

14. The method of claim 1, wherein the one or more values indicative of recombinant protein titre and/or quality comprises the recombinant protein titre measured by analytical Protein A chromatography, preferably on day 14 (±half day) of the fed batch culture.

15. The method of claim 1, wherein the one or more values indicative of recombinant protein titre and/or quality comprises the percentage of correctly assembled recombinant protein, measured preferably on day 14 (±half day) of the fed batch culture, e.g. by capillary electrophoresis sodium dodecyl sulphate (CE-SDS).

16. The method of claim 1, wherein the one or more values indicative of recombinant protein titre and/or quality comprises the titre of the main product, measured preferably on day 14 (±half day) of the fed batch culture, e.g. by quantitative size exclusion liquid chromatography coupled with mass spectrometry (qSEC-MS).

17. The method of claim 1, wherein the one or more values indicative of recombinant protein titre and/or quality is calculated by multiplying the value of recombinant protein titre as measured according to claim 14 by the percentage of correctly assembled recombinant protein as measured according to claim 15, wherein both measurements have been performed on the same day, preferably day 14 of the fed batch culture.

18. The method of claim 1, wherein the one or more values indicative of recombinant protein titre and/or quality is calculated by multiplying the value of recombinant protein titre as measured according to claim 13 by the percentage of correctly assembled recombinant protein as measured according to claim 15, wherein both measurements have been performed on the same day, preferably day 14 of the fed batch culture.

19. The method of claim 1, wherein the cells are mammalian cells, e.g. CHO cells.

20. The method of claim 1, wherein the recombinant protein is an antibody (e.g. an IgG antibody) or a fragment thereof.

21. The method of claim 1, wherein the amino acid sequence of the recombinant protein expressed by the plurality of candidate cell lines is the same.

22. The method of claim 1, wherein the machine-learning model comprises regression analysis, preferably a random forest regression model.

23. The method of claim 1, further comprising ranking the cell lines according to the predicted one or more values indicative of recombinant protein titre and/or quality, wherein the cell lines with higher predicted values are advanced to a next step of cell line screening or fermentation, e.g. a fed batch cell culture stage.

24. (canceled)

25. (canceled)

26. A non-transitory computer-readable medium having stored thereon computer readable instructions which, when executed by one or more processors, cause the one or more processors to carry out the method of claim 1.

27. A system comprising:
at least one processor; and
at least one non-transitory computer readable medium containing instructions that, when executed by the at least one processor, cause the at least one processor to perform the following steps:
(a) receive omics data for each of the plurality of candidate cell lines; and
(b) use a machine learning model to predict one or more values indicative of recombinant protein titre and/or quality,
wherein the machine learning model has been trained using a training dataset comprising omics data for a multiplicity of cell lines, and for each cell line, one or values indicative of recombinant protein titre and/or quality, and
wherein the omics data comprises:
i) transcriptomics data;
ii) metabolomics data;
iii) proteome data;
iv) transcriptomics and metabolomics data;
v) transcriptomics and proteome data;
vi) metabolomics and proteome data: or
vii) transcriptomics, metabolomics, and proteome data.
